(19) United States
(12) Patent Application Publication
(10) Pub. No.: US 2004/0005299 A1
(43) Pub. Date: Jan. 8, 2004

(54) Method for producing pseudo islets

(75) Inventors: Yin Liang, Bridgewater, NJ (US); Jian Zhu, North Haven, CT (US); Laurel Sweet, Branford, CT (US); James N. Livingston, Guilford, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT

(21) Appl. No.: 10/394,902

(22) Filed: Mar. 21, 2003

Related U.S. Application Data
(60) Provisional application No. 60/366,728, filed on Mar. 22, 2002.

(51) Int. Cl. A61K 45/00; C12N 5/08

(52) U.S. Cl. 424/93.7; 435/366

(57) ABSTRACT

The present invention relates to a method for preparing pseudo islets. In addition, the invention is also directed to methods of treating diabetes and diabetes-related disorders by administering compounds identified by the methods described herein.

This application claims benefit of U.S. Provisional Application Serial No. 60/366,728, filed on Mar. 22, 2002, the contents of which are incorporated herein by reference in their entirety.

### FIELD OF THE INVENTION

The present invention relates to a method for preparing functional pseudo islets. In addition, the invention is also directed to methods of treating diabetes and diabetes-related disorders by administering compounds identified by the methods described herein.

### BACKGROUND OF THE INVENTION

Diabetes is characterized by impaired glucose metabolism manifesting itself among other symptoms by an elevated blood glucose level in the diabetic patient. Underlying defects lead to the classification of diabetes into two major groups: Type 1 diabetes, or insulin dependent diabetes mellitus (IDDM), which arises when patients lack β-cells (β-cells produce insulin in the pancreatic gland), and Type 2 diabetes, or non-insulin dependent diabetes mellitus (NIDDM), which occurs in patients with an impaired β-cell function and alterations in insulin action.

Type 2 diabetes, is the more common form of diabetes, with 90-95% of hyperglycemic patients experiencing this form of the disease. The pathogenesis of Type 2 diabetes is characterized by insulin resistance and insulin insufficiency. Insulin resistant patients maintain euglycemia and do not develop overt diabetes provided that the pancreatic β-cells retain the capacity to release a sufficient amount of insulin to compensate for the insulin resistance. However, the β-cells are unable to maintain this high output of insulin, and eventually, the glucose-induced insulin secretion falls, leading to the deterioration of glucose homeostasis and to the subsequent development of overt diabetes. This hyperinsulinemia is also linked to insulin resistance, hypertriglyceridemia, low high-density lipoprotein (HDL) cholesterol, and increased plasma concentration of low-density lipoproteins (LDL). The association of insulin resistance and hyperinsulinemia with these metabolic disorders has been termed “Syndrome X,” and has been strongly linked to an increased risk of hypertension and coronary artery disease.

One approach to the pharmaceutical treatment of diabetes is to improve pancreatic islet function, in particular to enhance glucose-stimulated insulin release. That is, drugs that produce glucose-dependent insulin secretion would lead to a decrease in hyperglycemia, which is associated with the disease, without causing hypoglycemia or causing inappropriately high insulin levels. However, the identification and development of such insulinotropic compounds requires a high-efficiency, robust method for evaluating glucose-dependent insulin release.

In addition to affecting insulin secretion, another approach for the treatment of diabetes (e.g., Type 1, Type 2, impaired glucose tolerance) may be the preservation or restoration P-cell mass. Recent studies have shown that GLP-1 and analogs such as exendin-4, cause expansion of β-cell mass by increasing β-cell neogenesis and proliferation (De Leon et al., Diabetes 52:365-371, 2003; Farilla et al., Endocrinology 143:4397-4408, 2002). Drugs that effect β-cell mass by effecting apoptosis, neogenesis, or proliferation could be useful medicaments for the treatment of Type 1 and Type 2 diabetes. Thus, identification of drugs that increase insulin biosynthesis would be beneficial as a treatment for diabetes and related disorders by preventing or delaying β-cell failure.

At present, most cultured pancreatic β-cell lines do not respond to physiologic concentrations of glucose and therefore, are not suitable for testing compounds targeting glucose-regulated insulin release. Isolated primary pancreatic islets retain glucose-responsiveness; however, the utility of these cells is limited by: (1) the heterogeneous nature of pancreatic islets which induce large variations between groups of islets; and (2) the limited number of functional islets that may be isolated.

Some effort has been made to reduce the variation among pancreatic islets by using trypsin to disperse pancreatic islet tissue into single cells. However, these dispersed islet cells lose the ability to release insulin in response to glucose and other secretagogues. It has been determined that some cell-cell interaction is required for function. Therefore, re-aggregation of dispersed islet cells may be a means to recover topographic structure and to restore physiologically regulated insulin release.

Re-aggregation of dispersed islet cells to form pseudo islets may be achieved by a number of methods such as, culturing islet cells for several days (Weir et al., Metabolism 33:447-453, 1984), mechanically rotating the cells for 2 hours (Pipeleers et al., Endocrinology 117:806-816, 1985), or by mixing the cells with beads (Hopcroft et al., Endocrinology 117:2073-2080, 1985). However, these methods are time-consuming and result in large variations both in the size and yield of pseudo islets. Due to these limitations, these methods are not suitable for drug discovery.

The present invention provides a novel method to produce a homogenous population of pseudo islets. The method of the present invention may also be used to screen compounds targeting pancreatic β-cells, and to evaluate the potency of these compounds on insulin release.

### SUMMARY OF THE INVENTION

The present invention provides a novel method for preparing a homogenous population of pseudo islets. In one embodiment, the method of the present invention comprises the steps of subjecting pancreatic islets to an enzyme digest and seeding the dispersed islets into a vessel where the surface area of the vessel decreases from the top of the vessel to the bottom of the vessel (e.g., “V-bottom” plate, centrifuge tube, conical tube). In another embodiment, the method for preparing pseudo islets comprises the additional step of centrifugation. Specifically, the dispersed islet cells are centrifuged following the addition of the islet cells to the vessel. In a further embodiment, the enzymes used to digest the pancreatic islets comprise, for example, trypsin and DNAse I. In another aspect of the present invention, the digested pancreatic islet cells may be filtered prior to seeding into the vessel.

In another aspect of the invention, the pseudo islets may be isolated from fresh or frozen pancreatic islets. Furthermore, the pseudo islets may be isolated from any animal, including mammals.

The pseudo islets prepared by the method of the present invention may be used for a number of assays. In one aspect, the pseudo islets may be used, for example, to screen and evaluate insulinotropic or other compounds. In another embodiment, the pseudo islets may be used, for example, to measure insulin content, and to analyze insulin biosynthesis. In a further embodiment, the pseudo islets prepared by the method of the present invention may be used to characterize the effects of a compound on second messenger activity (e.g., cAMP, inositol triphosphate (IP₃), calcium). A further embodiment of the present invention relates to the use of pseudo islets to measure the metabolites of islet cells. In addition, the pseudo islets of the method of the present invention may be utilized to measure glucagon and somatostatin release and the regulation of glucagon and somatostatin by various compounds.

In a further aspect of the invention, the pseudo islets may be co-cultured with other cell types (e.g., fibroblasts).

The invention also relates to methods of treating diabetes and diabetes-related disorders by administering compounds identified by the methods described herein to a patient in need thereof.

Another embodiment of the present invention relates to kits for the preparation of pseudo islets. In one embodiment, the kit may comprise, for example, digestion enzymes and a vessel (e.g., “V-bottom” plates). In another embodiment, the kit may also include, for example, filters (e.g., nylon filters) and buffer solutions.

### DESCRIPTION OF THE DRAWINGS

FIG. 1. FIG. 1 represents a characterization of the pseudo islets prepared by the method of the present invention. Specifically, pseudo islets were incubated with glucose (panel A), acetylcholine (panel B), GLP-1 (panel C), and glybenclamide (panel D), and the effects of these compounds on insulin release were evaluated.

FIG. 2. FIG. 2 represents a characterization of the pseudo islets prepared by the method of the present invention. In particular, pseudo islets were incubated with insulin release secretagogues (forskolin and IBMX) and insulin release inhibitors (somatostatin and norepinephrine), and the effects of these compounds on insulin release were evaluated.

FIG. 3. FIG. 3 represents characterization of the pseudo islets co-cultured with fibroblasts. Pseudo islets were co-cultured with fibroblasts and incubated with increasing amounts of GLP-1 and the effects of co-culturing on insulin release were evaluated.

FIG. 4. FIG. 4 represents evaluation of insulin secretion by unknown compounds using the pseudo islets prepared by the method of the present invention. In particular, pseudo islets were incubated with unknown compounds in the presence or absence of GLP-1, and the effects of these compounds on insulin release were evaluated.

### DESCRIPTION OF THE INVENTION

It is to be understood that this invention is not limited to the particular methodology, protocols, cell lines, animal species or genera, and reagents described and as such may vary. It is also to be understood that the terminology used herein is for the purpose of describing particular embodiments only, and is not intended to limit the scope of the present invention which will be limited only by the appended claims.

It must be noted that as used herein, the singular forms “a,” “and,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs.

Definitions

For convenience, the meaning of certain terms and phrases employed in the specification, examples, and claims are provided below.

The term pancreatic islet refers to any group of small slightly granular endocrine cells that form anastomosing trabeculae among the tubules and alveoli of the pancreas and secrete insulin, glucagon, and somatostatin.

Pseudo islet refers to dispersed pancreatic islet cells that have been re-aggregated.

Intact islet refers to isolated pancreatic islets that maintain the natural topography.

The term insulinotropic refers to stimulating or affecting the production and activity of insulin.

The term animal includes all mammals such as rodents (e.g., rats, mice, guinea pigs, hamster), primates including humans and monkeys, sheep, canines (e.g., dogs), felines, bovines, and swine (e.g., pig).

The term vessel refers to any container where the surface area of the container decreases from the top of the container to the bottom of the container. For example, a vessel may be, but not limited to, a “V”-bottom plate, “U”-bottom plate, centrifuge tube, a conical tube, culture tube, 96-well plate, 384-well plate).

The term compound may include, but is not limited to, agonists, antagonists, small molecules, and antibodies. For example, the term “agonist” is meant to refer to an agent that mimics or up-regulates (e.g., potentiates or supplements) the biological activity of a protein. An agonist may be a wild-type protein or derivative thereof having at least one biological activity of the wild-type protein. An agonist may also be a compound that up-regulates expression of a gene or which increases at least one biological activity of a protein. An agonist can also be a compound which increases the interaction of a polypeptide with another molecule, for example, a target peptide or nucleic acid.

“Antagonist” is meant to refer to an agent that down-regulates (e.g., suppresses or inhibits) at least one biological activity of a protein. An antagonist may be a compound which inhibits or decreases the interaction between a protein and another molecule, for example, a target peptide or enzyme substrate. An antagonist may also be a compound that down-regulates expression of a gene or which reduces the amount of expressed protein present.

“Small molecule” refers to nucleic acids, peptides, polypeptides, peptidomimetics, carbohydrates, lipids, or other organic or inorganic molecules.

The term “antibody” is intended to include whole antibodies, for example, of any isotype (IgG, IgA, IgM, IgE, etc.), and includes fragments thereof. Antibodies may be fragmented using conventional techniques and the fragments screened for utility. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The invention includes polyclonal, monoclonal, or other purified preparations of antibodies and recombinant antibodies.

The present invention describes the preparation of a homogenous population of pseudo islets and its application in the research and development of insulinotropic compounds.

To date, multiple efforts using islet cells in cell culture plates to study insulin release have failed due to two major reasons: 1) loss of the topographic structure of pancreatic islets and thus, loss of the response to insulin secretagogues; and 2) the lack of attachment of these cells to the plates after prolonged culture.

To overcome these difficulties, the present invention provides a novel approach utilizing an islet cell incubation method. The critical step of the method of this invention is to seed dispersed islet cells into a vessel where the surface area of the vessel decreases from the top of the vessel to the bottom of the vessel (e.g., “V-bottom” plate). A pseudo islet is then generated following centrifugation. An advantage of using this type of vessel is that the dispersed islet cells are collected at the bottom of the vessel forming a cell cluster. This cluster of cells forms a pseudo islet. This cell collection step cannot be accomplished in a flat bottom plate, because centrifugation will only disperse the cells along the bottom of the plate, and thus, the cells cannot form a pseudo islet.

Centrifugation is another key step for this pseudo islet method. After the dispersed cells are seeded in the vessel, the individual cells may spread unevenly across the bottom of the vessel. These uneven cell clusters may vary in size and therefore, may produce large variations in insulin release. The combination of a vessel where the surface area of the vessel decreases from the top of the vessel to the bottom of the vessel and centrifugation permit the generation of similar pseudo islets. Thus, this method of pseudo islet preparation is highly efficient and robust. Following an overnight culture, insulin release is restored and the pseudo islets are responsive to glucose and other secretagogues. In addition, this method minimizes cell loss during changes of medium which may occur several times during an experiment.

As compared to the classical static islet incubation method, there are two major advantages to the islet cell method of the present invention. First, this method significantly reduces the variation of insulin release from intact islets. Pancreatic islets consist of α-, β-, and δ-cells and the composition of these cell types varies between different islets. In addition, the total number of cells for each islet may range from 1,000-10,000 cells. Thus, islets are an organ of heterogeneity. Although islets were treated under the same conditions, it has been demonstrated that the heterogeneity of islets produces a large variation in insulin release between different islet (Hopcroft et al., Hormon. Metab. Res. 17:559-561, 1985; Colella et al., Life Sci. 37:1059-1065, 1985). This variation markedly limits the use of static islet incubation in the pharmaceutical industry. In the method of the present invention, trypsin is used to disperse islet tissue into individual cells, and these islet cells are then seeded into a vessel to form pseudo islets. Accordingly, the heterogeneity among islets is overcome, and thus, significantly improving the quality of each experiment.

Secondly, the method of the present invention significantly increases assay capability. The classical static islet incubation requires adding at least 5 islets to each incubation well (Liang et al., Biochim. Biophys. Acta 1405:1-13, 1998), whereas the method of the present invention requires only 2,500 islet cells (which is about the same size as a small islet). Furthermore, using intact islets requires groups of islets of similar size and thus, limits the size of a study because only a portion of isolated islets may been used. However, the method of the present invention utilizes dispersed islets cells and thus, all isolated islets may be used in study. In addition, the method of the present invention avoids manual selection of every islet and therefore, saves considerable time on sample preparation.

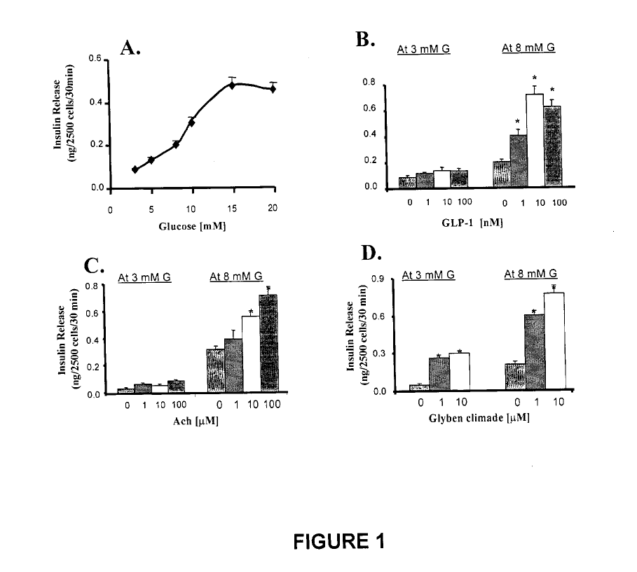

Overall, the method of the present invention provides an improvement over currently used islet cell purification methods, and markedly increases the efficiency of islet cell preparation and the assay capacity of a particular experiment. This novel method has been used to characterized compounds and peptides that activate or inhibit insulin secretion in the classical pancreatic islet system. For example, glucose is the primary physiological regulator for insulin release from pancreatic β-cells. When blood glucose levels are less than or equal to 5 mM, basal insulin release is very low. However, increasing blood glucose levels from 5 mM to 15 mM will significantly enhance plasma insulin levels due to increased insulin release. This glucose responsiveness is very well-preserved in isolated pancreatic islets and serves as a key criterion to assess the quality of the islet preparation and static islet incubation. Using the method of the present invention, dispersed islet cells were incubated with glucose at concentrations ranging from 2.5-20 mM. Insulin release from β-cells was enhanced gradually following the increase in glucose concentrations of the medium, and reached a plateau at 15 mM glucose (FIG. 1, panel A). This result indicates that pseudo islets prepared by the dispersed islet cell method of the present invention preserves glucose responsiveness and insulin release.

In additional studies, the effects of acetylcholine, GLP-1, and glybenclamide on insulin release were analyzed using pseudo islets prepared by the method of the present invention. Acetylcholine (Ach) is a neurotransmitter that stimulates insulin release in a glucose-dependent manner, and GLP-1 is an incretin that potentiates glucose-stimulated insulin release. Dispersed islet cells prepared by the method of the present invention were incubated with media containing 8 mM glucose and either Ach or GLP-1. A significant stimulatory effect on insulin secretion was observed in the presence of Ach and GLP-1 with EC₅₀ values of 10.1 μM and 4.8 μM, respectively (FIG. 1, panel B and C). This data demonstrates that dispersed islet cells prepared by the method of the present invention display a similar response to Ach and GLP-1 as compared with responses observed in intact islets (Gilon et al., Endocr. Rev. 22:565-604, 2001; Siege et al., Eur. J. Clin. Invest. 29:610-614, 1999).

Glybenclamide stimulates insulin release by blocking K_{ATP} channels and increasing intracellular calcium levels. This insulinotropic effect occurs at both low (3 mM) and high (≧8 mM) glucose concentrations. This characteristic effect is well documented in intact islet studies (Boyd et al., Am. J. Med. 89:3S-10S, 1990). Dispersed islet cells prepared by the method of the present invention demonstrate an insulin release response similar to responses reported in the literature (Sako et al., Metabolism 35:944-949, 1986). The EC₅₀ of glybenclamide on insulin release at a glucose concentration of 8 mM is 0.37 μM (FIG. 1, panel D).

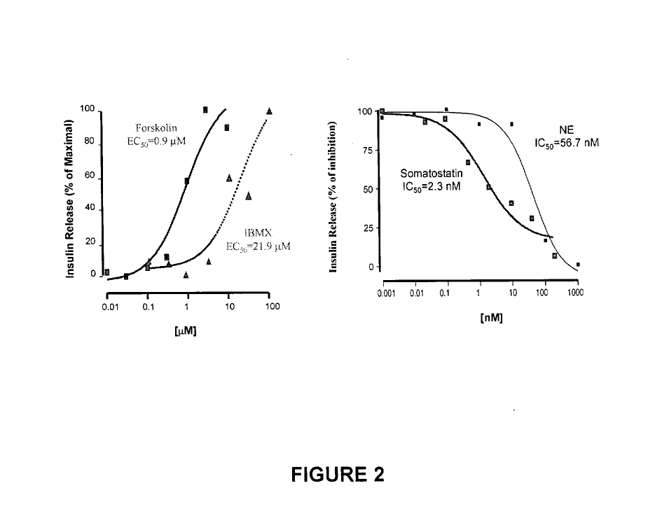

The cellular second messenger, cyclic AMP (cAMP) also plays an important role in glucose-stimulated insulin release. In studies using intact islets, both forskolin and IBMX increase cAMP content in pancreatic islet tissue and stimulate insulin release (Gromada et al., Pflugers Arch. 435:583-594, 1998; Ammon et al., Naunyn Schmiedebergs Arch. Pharmacol. 326:364-367, 1984; Ziegler et al., Acta Biol. Med. Ger. 41:1171-1177, 1982). Dispersed islet cells prepared by the method of the present invention demonstrate a similar forskolin and IBMX effect on insulin release (FIG. 2, panel A). The EC₅₀ of forskolin and IBMX on insulin release of dispersed islet cells in the presence of glucose at a concentration of 8 mM is 0.9 μM and 21.9 μM, respectively.

Glucose-stimulated insulin release is strongly inhibited by the neurotransmitter norepinephrine (NE) or by somatosotain, an intestinal hormone, and has been well documented in intact islet studies (Yamazaki et al., Mol. Pharmacol. 21:648-653, 1982; Claro et al., Acta Endrocrinol. (Copenh) 85:379-388, 1977). Dispersed islet cells prepared by the method of the present invention demonstrate that both NE and somatostain inhibit glucose-stimulated insulin release in a dose-dependent manner with IC₅₀s of 56.7 nM and 0.9 nM, respectively (FIG. 2, panel B).

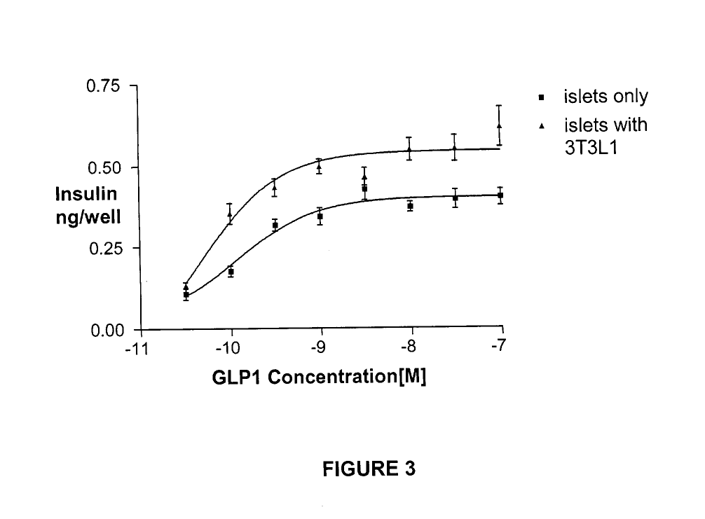

Islet β-cell survival and function in tissue culture can be promoted by co-culture of the islets with other cells and has been reported for islet cell monolayer cultures (Rabinovitch et al., Diabetes 28 (12):1108-13, 1979). Dispersed islet cells prepared by the method of the present invention demonstrate enhanced GLP-1 mediated insulin secretion when the pseudo islets are co-cultured with fibroblasts (FIG. 3).

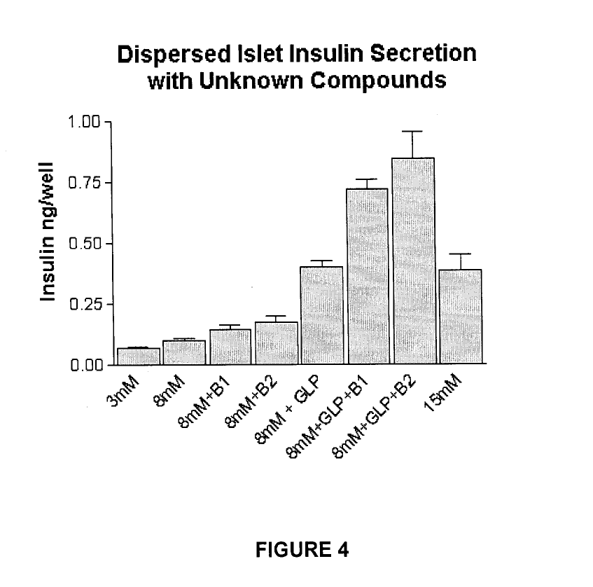

Insulinotropic compounds may be evaluated in dispersed islets prepared by the method of the present invention for their ability to potentiate insulin secretion in the presence of glucose, and in the presence and absence of GLP-1 (FIG. 4).

In summary, the results of the studies described above verify that dispersed islet cells prepared by the method of the present invention are similar to data using intact pancreatic islets. Hence, dispersed islet cells prepared by the method of the present invention are suitable for replacing the classical static islet incubation method, and may be used to screen and evaluate insulinotropic compounds. In addition, pseudo islets prepared by the method of the present invention may also be utilized to measure insulin content, to examine insulin biosynthesis, to test the effects of a compound on, for example, cAMP (e.g., Direct SPA Screening Biotrak Assay Kit, Amersham, Piscataway, N.J.), as well as to measure metabolites in islet cells (e.g., spectrometric or fluorometric enzyme assays, or any other method known to those skilled in the art).

Furthermore, there is a significant number of α-cells in the pseudo islets prepared by the method of the present invention. Thus, these pseudo islet cells may also be used to measure glucagon release. Hyperglucagonemia is a common phenomenon in Type 2 diabetes. The major physiological effect of glucagon is to increase hepatic glucose production. An enhancement of circulating glucagon levels in Type 2 diabetic patients contributes significantly to fasting hyperglycemia. Thus, inhibition of glucagon release or reduction of the glucagon effect on target tissue is another approach to treat diabetes. The dispersed islet cells prepared by the method of the present invention provide a robust method to measure glucagon release and its regulation by a variety of compounds.

The method of the present invention may be used to identify compounds that are effective in the treatment of Type 2 diabetes mellitus (including associated diabetic dyslipidemia and other diabetic complications), as well as other diabete-related disorders such as hyperglycemia, hyperinsulinemia, impaired glucose tolerance, impaired fasting glucose, dyslipidemia, hypertriglyceridemia, Syndrome X, insulin resistance, obesity, atherosclerotic disease, hyperlipidemia, hypercholesteremia, low HDL levels, hypertension, cardiovascular disease (including atherosclerosis, coronary heart disease, coronary artery disease, and hypertension), cerebrovascular disease, peripheral vessel disease, lupus, polycystic ovary syndrome, carcinogenesis, and hyperplasia.

Demonstration of the activity of compounds identified by the method of the present invention may be accomplished through a number of in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia or atherosclerotic disease and related disorders such as hypertriglyceridemia and hypercholesteremia, the following assays may be used.

Method for Measuring Blood Glucose Levels. db/db mice (obtained from Jackson Laboratories, Bar Harbor, Me.) are bled (by either eye or tail vein) and grouped according to equivalent mean blood glucose levels. They are dosed orally (by gavage in a pharmaceutically acceptable vehicle) with the test compound once daily for 14 days. At this point, the animals are bled again by eye or tail vein and blood glucose levels were determined. In each case, glucose levels are measured with a Glucometer Elite XL (Bayer Corporation, Elkhart, Ind.).

Method for Measuring Triglyceride Levels. hApoA1 mice (obtained from Jackson Laboratories, Bar Harbor, Me.) are bled (by either eye or tail vein) and grouped according to equivalent mean serum triglyceride levels. They are dosed orally (by gavage in a pharmaceutically acceptable vehicle) with the test compound once daily for 8 days. The animals are then bled again by eye or tail vein, and serum triglyceride levels are determined. In each case, triglyceride levels are measured using a Technicon Axon Autoanalyzer (Bayer Corporation, Tarrytown, N.Y.).

Method for Measuring HDL-Cholesterol Levels. To determine plasma HDL-cholesterol levels, hApoA1 mice are bled and grouped with equivalent mean plasma HDL-cholesterol levels. The mice are orally dosed once daily with vehicle or test compound for 7 days, and then bled again on day 8. Plasma is analyzed for HDL-cholesterol using the Synchron Clinical System (CX4) (Beckman Coulter, Fullerton, Calif.).

Method for Measuring Total Cholesterol, HDL-Cholesterol, Triglycerides, and Glucose Levels. In another in vivo assay, obese monkeys are bled, then orally dosed once daily with vehicle or test compound for 4 weeks, and then bled again. Serum is analyzed for total cholesterol, HDL-cholesterol, triglycerides, and glucose using the Synchron Clinical System (CX4) (Beckman Coulter, Fullerton, Calif.). Lipoprotein subclass analysis is performed by NMR spectroscopy as described by Oliver et al., (Proc. Natl. Acad. Sci. USA 98:5306-5311, 2001).

Method for Measuring an Effect on Cardiovascular Parameters. Cardiovascular parameters (e.g., heart rate and blood pressure) are also evaluated. SHR rats are orally dosed once daily with vehicle or test compound for 2 weeks. Blood pressure and heart rate are determined using a tail-cuff method as described by Grinsell et al., (Am. J. Hypertens. 13:370-375, 2000). In monkeys, blood pressure and heart rate are monitored as described by Shen et al., (J. Pharmacol. Exp. Therap. 278:1435-1443, 1996).

Based on the methods described above, or other well known assays used to determine the efficacy for treatment of conditions identified above in mammals, and by comparison of these results with the results of known medicaments that are used to treat these conditions, the effective dosage of a compound can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound may be ascertained by those skilled in the art using conventional treatment tests.

The compounds identified by the methods of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a pharmaceutically effective amount of a compound identified by the methods described herein. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is that amount which produces a result or exerts an influence on the particular condition being treated. The compounds identified by the methods described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds identified by the methods of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds identified by the methods of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1, 1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC-CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds identified by the methods described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds identified by the methods described herein may also be utilized in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000)

The following examples are presented to illustrate the invention described herein, but should not be construed as limiting the scope of the invention in any way.

Capsule Formulation
A capsule formula is prepared from:

| | |
|---|---|
| Active ingredient | 40 mg |
| Starch | 109 mg |
| Magnesium stearate | 1 mg |

The components are blended, passed through an appropriate mesh sieve, and filled into hard gelatin capsules.

Tablet Formulation
A tablet is prepared from:

| | |
|---|---|
| Active ingredient | 25 mg |
| Cellulose, microcrystaline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A 5 mg/ml solution of the active ingredient is made using sterile, injectable water, and the pH is adjusted if necessary. The solution is diluted for administration to 1-2 mg/ml with sterile 5% dextrose and is administered as an IV infusion over 60 minutes.

Intramuscular suspension
The following intramuscular suspension is prepared:

| | |
|---|---|
| Active ingredient | 50 mg/ml |
| Sodium carboxymethylcellulose | 5 mg/ml |
| TWEEN 80 | 4 mg/ml |
| Sodium chloride | 9 mg/ml |
| Benzyl alcohol | 9 mg/ml |

The suspension is administered intramuscularly.

Hard Shell Capsules

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### EXAMPLES

The present invention is further illustrated by the following examples which should not be construed as limiting in any way. The contents of all cited references (including literature references, issued patents, published patent applications, and co-pending patent applications) cited throughout this application are hereby expressly incorporated by reference.

### Example 1

### Preparation of Pseudo Islets in 96-Well Plates

Pancreata from four Sprague Dawley rats were divided into small pieces approximately 1 mm² or smaller in size. The tissue was then rinsed three times with Hanks-Hepes buffer (127 mM NaCl, 5.4 mM KCl, 0.34 mM Na₂HPO₄, 4.4 mM KH₂PO4, 20 mM HEPES, 1.2 mM CaCl₂/5 mM glucose), and digested with collagenase (Liberase, 0.25 mg/ml, Roche Diagnostic Corp., Indianapolis, Ind.) at 37° C. in a water bath shaker for 10 minutes.

The digested pancreata tissue was rinsed three times with 50 ml of Hanks-Hepes buffer to remove the collagenase. The tissue pellet was then filtered through a 250 μm filter and the filtrate was mixed with 16 ml of 27% Ficoll (Sigma, St. Louis, Mo.) w/v in Hanks-Hepes buffer. Three layers of Ficoll (23%, 20.5%, and 11%, respectively; 8 ml of each concentration) were then loaded on top of the mixture of islet tissue in 27% Ficoll to form a gradient.

The Ficoll gradient was then centrifuged at 1,600 rpm for 10 minutes at room temperature. The pancreatic islets were concentrated at the interphase between 11% and 20.5%, and between 20.5% and 23% depending on the size of islets. The islets were collected from the two interphases and rinsed twice with Ca⁺⁺-free Hanks-Hepes buffer. The islets were then suspended in 5 ml Ca⁺⁺-free Hanks-Hepes buffer containing 1 mM EDTA and incubated for 8 minutes at room temperature.

Trypsin and DNAse I were added to the islet suspension for a final concentration of 25 μg/ml and 2 μg/ml, respectively. This suspension was incubated with shaking at 30° C. for 10 minutes. The trypsin digestion was stopped by adding 40 ml RPMI 1640 (GIBCO Life Technologies, Invitrogen, Carlsbad, Calif.) with 10% FBS. The trypsin digested islet cells were then filtered through a 63 μm nylon filter (PGC Scientific, Frederick, Md.) to remove large cell clusters.

The dispersed islet cells were then washed, counted using hemacytometer under the microscope, and seeded into “V-bottom” 96-well plates (2,500 cells per well). However, a range of 1,000 to 10,000 cells per well may used. The dispersed islet cell suspension was then centrifuged at 1,000 rpm for 5 minutes. The Hanks-Hepes buffer was removed and replaced with 200 μl RPMI 1640 medium containing 10% FBS, 1% Penicillin-Streptomycin, and 2 mM L-glutamine. Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% CO₂, and then used for assays.

### Example 2

### Pseudo Islet Incubation with Fibroblasts

Dispersed islet cells (prepared by the method described in Example 1) were washed with regular RPMI 1640 medium with 10% FBS, counted using hemacytometer under the microscope, and seeded into “V-bottom” 96-well plates with fibroblasts (2,500 islet cells and 1,250 fibroblasts cells per well). The cell suspension was then centrifuged at 1,000 rpm for 5 minutes to collect the dispersed islet cells concentrated at the V-bottom of the plate forming pseudo islets. These pseudo islets were then co-cultured with the fibroblasts cells overnight in a cell culture incubator at 37° C. with 5% CO₂, and then used for assays.

### Example 3

### Freezing and Thawing of Pseudo Islets

Dispersed islet cells (prepared by the method described in Example 1) were counted as described above and diluted in regular RPMI 1640 medium with 10% FBS and 10% DMSO to a concentration of 2×10⁵ cells per ml. An aliquot (1 ml) was transferred to a cryotube and the cryotube was placed in a rack in the vapor phase in a liquid nitrogen tank prior to freezing in liquid nitrogen.

Cells were thawed and then washed with regular medium and seeded into “V-bottom” 96-well plates (5,000 cells per well). Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% C02, and then used for assays.

### Example 4

### Static Pseudo Islet Incubation for Insulin Release Assay

Pseudo islets were prepared by the method described in Example 1. Following an overnight incubation, the RPMI 1640 medium was removed and replaced by 100 μl Krebs-Ringer-Hepes buffer (115 mM NaCl, 5.0 mM KCl, 24 mM NaHCO₃, 2.2 mM CaCl₂, 1 mM MgCl₂, 20 mM HEPES, 0.25% BSA, 0.002% Phenol Red, pH 7.35-7.40). The cell suspension was then centrifuged for 5 minutes at 1,000 rpm to pellet the dispersed islet cells.

Pseudo islets in 96-well plates were incubated in a water bath at 37° C. continuously gassed with 95% O₂/5% CO₂ for pre-incubation for 30 minutes. The pre-incubation buffer was removed and replaced with 50 μl incubation buffer (Krebs-Ringer-Hepes buffer, pH 7.35-7.40) containing various test substrates.

The 96-well plate was centrifuged again at 1,000 rpm for 5 minutes to form pseudo islets. These pseudo islets in 96-well plates were statically incubated in a water bath at 37° C. continuously gassed with 95% O₂/5% CO₂ for 60 minutes. The incubation buffer (25 μl) was collected after the 60-minute incubation and used for an insulin content assay (ELISA assay, ALPCO, N.H.).

### Example 5

### Static Pseudo Islet Incubation for Insulin Biosynthesis

Pseudo islets are prepared as described in Example 1. After an overnight culture, the pseudo islets are preincubated in KRBH (135 mM NaCl, 3.6 mM KCl, 10 mM HEPES, 5 mM NaHCO₃, 0.5 mM NaH₂PO₄, 0.5 mM MgCl₂, 1.5 mM CaCl₂, 0.1% Bovine Serum Albumin) containing 3 mM glucose for 30 minutes at 37° C., and then incubated for 90 minutes at 37° C. with test compounds and 2 μM ³H-Leucine (100 μL) (Amersham, Piscataway, N.J.). The pseudo islets are then washed 3× with KRBH containing 1 mM leucine (Sigma, St. Louis, Mo.), lysed in 2 mM acetic acid (100 μl), sonciated for 15 seconds, and neutralized with 10 N NaOH (20 μl). HEPES (50 mM) containing 0.1% Triton X-100 is added to bring the volume to 1 ml and the samples are spun for 10 minutes at 1750×g. Protein A Agarose (50 μl per sample) is preincubated with anti-insulin antibody (Linco, St. Charles, Mo.) (100 μl per sample) for 2 hours and washed twice. The antibody bead mixture (50 μl) was added to 750 μl of sample and incubated overnight at 4° C. The immunoprecipitates are washed 3× with HEPES (50 mM) containing 0.1% Triton X-100. The beads are then counted in a scintillation counter.

### Example 6

### Static Pseudo Islet Incubation for Glucagon Release

Pseudo islets are prepared as described in Example 1. Following an overnight incubation, the RPMI 1640 medium was removed and replaced by 100 μl Krebs-Ringer-Hepes buffer (115 mM NaCl, 5.0 mM KCl, 24 mM NaHCO₃, 2.2 mM CaCl₂, 1 mM MgCl₂, 20 mM HEPES, 0.25% BSA, 0.002% Phenol Red, pH 7.35-7.40). The cell suspension was then centrifuged for 5 minutes at 1,000 rpm to pellet the dispersed islet cells.

Pseudo islets in 96-well plates were incubated in a water bath at 37° C. continuously gassed with 95% O₂/5% CO₂ for pre-incubation for 30 minutes. The pre-incubation buffer was removed and replaced with 50 μl incubation buffer (Krebs-Ringer-Hepes buffer, pH 7.35-7.40) containing various test compounds.

The 96-well plate was centrifuged again at 1,000 rpm for 5 minutes to form pseudo islets. These pseudo islets in 96-well plates were statically incubated in a water bath at 37° C. continuously gassed with 95% O₂/5% CO₂ for 60 minutes. The incubation buffer (25 μl) was collected after the 60-minute incubation and used for a glucagon content assay (Glucagon RIA kit; Linco, St. Charles, Mo.).

### Example 7

### Assay for Identifying Insulinotropic Compounds

Pseudo islets were prepared as described in Example 1. The dispersed islet cells were then washed, counted using a hemacytometer, and seeded into “V-bottom” 96-well plates (2,500 cells per well) with 200 μl RPMI 1640 medium containing 10% FBS, 1% Penicillin-Streptomycin, and 2 mM L-glutamine. Next, the 96-well plates were centrifuged at 1,000 rpm for 5 minutes to collect the dispersed islet cells concentrated at the V-bottom of the plate forming pseudo islets. These pseudo islets were then cultured overnight in a cell culture incubator at 37° C. with 5% CO₂.

Following the overnight incubation, the RPMI 1640 medium was removed and replaced by 100 μl Krebs-Ringer-HEPES buffer (115 mM NaCl, 5.0 mM KCl, 24 mM NaHCO₃, 2.2 mM CaCl₂, 1 mM MgCl₂, 20 mM HEPES, 0.25% BSA, 0.002% Phenol Red, pH 7.35-7.40) with 3 mM glucose. The cell suspension was then centrifuged for 5 minutes at 1,000 rpm to pellet the dispersed islet cells.

The pseudo islets in 96-well plates were incubated in a water bath at 37° C. continuously gassed with 95% O₂/5% CO₂ for a pre-incubation of 30 minutes. The pre-incubation buffer was removed and replaced with 50 μl incubation buffer (Krebs-Ringer-HEPES buffer, pH 7.35-7.40) containing the test compounds. The 96-well plates were centrifuged again at 1,000 rpm for 5 minutes to form pseudo islets. These pseudo islets were then statically incubated in a water bath at 37° C. continuously gassed with 95% O₂/5% CO₂ for 30 minutes. The incubation buffer (25 μl) was collected after the 30-minute incubation and used for an insulin content assay.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the field are intended to be within the scope of the following claims.

We claim:

1. A method for preparing pseudo islets comprising the steps of
treating pancreatic islets with an enzyme digest; and
seeding the digested islets into a vessel where the surface area of the vessel decreases from the top of the vessel to the bottom of the vessel.

2. The method of claim 1 further comprising the step of centrifuging said islets to aggregate the islets.

3. The method of claim 2 further comprising the step of freezing said peudo islets.

4. The method of claim 1, wherein said enzymes are trypsin, DNase I, or dispase.

5. The method of claim 1, wherein said digested islets are filtered prior to seeding.

6. The method of claim 1, wherein said pseudo islets are isolated from mammalian pancreatic tissue.

7. The method of claim 5, wherein said mammalian pancreatic tissue is human.

8. The method of claim 5, wherein said pseudo islets are isolated from fresh pancreatic tissue.

9. The method of claim 5, wherein said pseudo islets are isolated from frozen pancreatic tissue.

10. The method of claim 1, wherein said vessel is a V-bottom plate.

11. The method of claim 1 further comprising the step of co-culturing the pseudo islets with fibroblast cells.

12. A method of identifying insulinotropic compounds comprising the steps of
isolating pseudo islets by the method of claim 1;
adding a test compound to the isolated pseudo islets; and
measuring the effect of said compound on insulin secretion.

13. A method of treating diabetes or diabetes-related disorders administering to a patient in need thereof an effective amount of a compound identified by the method of claim 10.

14. The method of claim 11, wherein said diabetes-related disorders are selected from the group consisting of hyperglycemia, hyperinsulinemia, impaired glucose tolerance, impaired fasting glucose, dyslipidemia, hypertriglyceridemia, Syndrome X, insulin resistance, obesity, atherosclerotic disease, hyperlipidemia, hypercholesteremia, low HDL levels, hypertension, cardiovascular disease, cerebrovascular disease, peripheral vessel disease, lupus, polycystic ovary syndrome, carcinogenesis, and hyperplasia.

15. A pharmaceutical composition comprising an effective amount of a compound identified by the method of claim 10 in combination with a pharmaceutically acceptable carrier.

16. A kit for preparing pseudo islets comprising digestion enzymes and a vessel where the surface area of the vessel decreases from the top of the vessel to the bottom of the vessel.

17. A method of analyzing insulin biosynthesis comprising the steps of
isolating pseudo islets by the method of claim 1;
adding a test compound to the isolated pseudo islets; and
measuring the effect of said compound on insulin content.

18. A method of treating diabetes or diabetes-related disorders administering to a patient in need thereof an effective amount of a compound identified by the method of claim 17.

19. A method of measuring glucagon release comprising the steps of
isolating pseudo islets by the method of claim 1;
adding a test compound to the isolated pseudo islets; and
measuring the effect of said compound on glucagon content.

20. A method of treating diabetes or diabetes-related disorders administering to a patient in need thereof an effective amount of a compound identified by the method of claim 19.

21. A method of measuring somatostatin release comprising the steps of
isolating pseudo islets by the method of claim 1;
adding a test compound to the isolated pseudo islets; and
measuring the effect of said compound on somatostatin content.

22. A method of treating diabetes or diabetes-related disorders administering to a patient in need thereof an effective amount of a compound identified by the method of claim 21.
